(19) United States
(12) Patent Application Publication
(10) Pub. No.: US 2003/0082189 A1
(43) Pub. Date: May 1, 2003

(54) Humanized neutralizing antibodies against hemolytic uremic syndrome

(75) Inventors: Saul Tzipori, Shrewsbury, MA (US); Ramaswamy Balakrishnan, Framingham, MA (US); Arthur Donohue-Rolfe, Sudbury, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: Trustees of Tufts College

(21) Appl. No.: 10/230,614

(22) Filed: Aug. 29, 2002

Related U.S. Application Data
(63) Continuation of application No. 09/302,125, filed on Apr. 29, 1999.
Application No. 09/302,125 is a division of application No. 08/749,704, filed on Nov. 15, 1996, now abandoned.

(51) Int. Cl. A61K 39/40

(52) U.S. Cl. 424/164.1

(57) ABSTRACT

Disclosed is a therapeutic method for the treatment of hemolytic uremic syndrome. More specifically, the method includes the administration of monoclonal antibodies, chimeric monoclonal antibodies and monospecific polyclonal antibodies specific for Shiga like toxin.

### BACKGROUND OF THE INVENTION

Since the first documented outbreaks in 1982, infections from Enterohemorrhagic Escherichia coli (EHEC) have been a major public health concern in the United States and in Europe. In the United States, an estimated 20,000 cases of EHEC annually result in death, with 200-500 of these cases occurring in children. EHEC infection typically results from the ingestion of under cooked beef, particularly hamburgers. Outbreaks of disease have been reported in association with consumption of hamburgers in fast food chains, in nursing homes, and in day-care centers. EHEC has been found to occur sporadically in children anywhere in the United States or Europe, while in adults EHEC outbreaks have shown a propensity for certain regions, for instance the western United States. In several states, infection with EHEC is a notifiable disease. Additionally, outbreaks of the disease have been found to occur at any time of the year. Infection with EHEC bacteria in adults can lead to bloody diarrhea which lasts several days. In children, however, the infection may lead, in addition to bloody diarrhea, to systemic complications which can be either fatal, due to acute renal failure and serious neurological involvement, or lead to permanent kidney damage. The kidney damage and the neurological symptoms which are caused by one of 2 toxins is known as hemolytic uremic syndrome (HUS). In children there is normally a prodromal period of 4 to 7 days between the bloody diarrhea and development of HUS. During this prodromal period an effective preventative treatment, if one was available, might prevent the development of HUS.

Currently there are three accepted characteristics of all EHEC strains. First, they all harbor a similar but not identical plasmid of about 60 mDa molecular size. The role of this plasmid is currently under investigation, but there are suggestions that it plays a role in adherence or at least control the expression of genes that are involved in adherence. Second, all EHEC strains are capable of attaching intimately to epithelial cells and effacing microvilli in the large intestine of newborn piglets and presumable in man. Thirdly, all EHEC produce toxins known as Shiga-like toxins. Shiga-like toxins are also referred to as verotoxins. Shiga-like toxins consist of one enzymatically active A chain and five B chains that are responsible for cell binding. The toxins are potent protein synthesis inhibitors and are particularly cytotoxic to both HeLa and Vero cells in culture. In the majority of EHEC strains, the toxin genes are carried on lysogenic phages. Based on antigenic relatedness to Shiga toxin, there are two general classes of Shiga-like toxins. Shiga-like toxin I is neutralized by antibody against Shiga toxin, the toxin produced by Shigella dysenteriae type I strains. Shiga-like toxin II is defined as toxin which is not neutralized by antibody directed against Shiga toxin. By amino acid comparison, SLT-I and SLT-II are 56% homologous. The two toxins have identical sets of glycolipid receptors and an identical mode of action. All EHEC strains isolated to date have been found to produce either one toxin or both. The role of toxin in the pathogenesis of both hemorrhagic colitis and hemolytic uremic syndrome is still not definitive. However, there is strong circumstantial evidence linking SLT II with HUS.

### SUMMARY OF THE INVENTION

The invention relates in one aspect to a therapeutic method to treat hemolytic uremic syndrome by administering to an individual a therapeutically effective amount of monoclonal antibody which binds specifically to either Shiga toxin, Shiga like toxin I or Shiga like toxin II. The hemolytic uremic syndrome is typically caused by an Enterohemorrhagic Escherichia coli. Shiga toxin which is identical to SLT-I is produced by Shigella sp.

In another aspect the invention relates to a monoclonal antibody which binds specifically to Shiga toxin, Shiga like toxin I or Shiga like toxin II. The monoclonal antibody is either a human monoclonal antibody or a chimeric monoclonal antibody. The monoclonal antibody can be produced by recombinant DNA methodology.

The invention relates in a another aspect to a therapeutic method to treat hemolytic uremic syndrome by administering to an individual a therapeutically effective amount of monospecific polyclonal antibodies which bind specifically to either Shiga toxin, Shiga like toxin I or Shiga like toxin II. The hemolytic uremic syndrome is caused by an Enterohemorrhagic Escherichia coli, and Shigella sp.

In another aspect, the invention relates to monospecific polyclonal antibodies which bind specifically to either Shiga toxin, Shiga like toxin I or Shiga like toxin II. The monospecific polyclonal antibodies are human monospecific antibodies.

### DETAILED DESCRIPTION OR THE INVENTION

The present invention is based, in one aspect, on the use of a therapeutic method to treat an individual suffering from hemolytic uremic syndrome (HUS) caused by a virulent strain of an Enterohemorraghic E. coli (EHEC). The treatment of HUS as disclosed herein involves the use of a monoclonal antibody, a cocktail of monoclonal antibodies or monospecific polyclonal antibodies, which specifically bind either Shiga toxin (ST), Shiga like toxin I (SLT-I) or Shiga like toxin II (SLT-II). In the present invention, monospecific polyclonal antibodies encompass antigen specific serum antibodies that are produced following immunization of an animal, and which are subsequently purified. Shiga toxin and Shiga like toxin (SLT) are composed of two unique chains, one A chain and five B chains, each encoded by a novel gene. The A chain contains the enzymatic activity, while the five B chains are responsible for cell binding. HUS is one clinical manifestation among several associated with SLT toxemia and is primarily found to afflict children and the elderly. The most common strain of EHEC found associated with outbreaks of HUS in the United States is Escherichia coli (E. coli) 0157:H7.

The use of antibodies to protect an individual from ST, SLT-I or SLT-II induced disease is described in more detail in the following section. The experiments described in the following section demonstrated, for example, that antibodies with specificity for SLT could be used to protect a mammal from cerebral hemorrhage and mortality following challenge with a virulent SLT producing bacterial strain. Although the bulk of the in vivo data reported herein were generated in experiments employing piglet indicator assays for protection against SLT-I and/or SLT-II, the fundamental principles are applicable to humans as well. The monoclonal and polyclonal antibodies of the present invention, which bind to either ST, SLT-I or SLT-II, are designed to protect a human individual against the pathologic effects of SLT produced by an EHEC, including HUS. Finally, based on the present disclosure, those of skill in the art will recognize that only routine experimentation will be necessary in order to permit them to rapidly identify monoclonal and polyclonal antibodies for application to the therapeutic treatment of human disease.

The present invention relates in one embodiment to methods for the treatment of an individual suffering from HUS. For example, passive immunization represents one therapeutic approach. Passive immunization can be accomplished using a prophylactically effective amount of a monoclonal antibody, a cocktail of monoclonal antibodies or monospecific polyclonal antibodies. Preferably, such passive immunization is generally accomplished prior to the onset or in the very early stages of the disease.

To treat HUS, a monoclonal antibody , a cocktail of monoclonal antibodies or a monospecific polyclonal antibodies should be given to the affected individual upon detection of the first indications of SLT toxemia. These initial symptoms include the presence of relatively large quantities of blood in diarrhea and bacterial shedding into the feces. If the treatment of HUS is delayed, the amount of a monoclonal antibody, a cocktail of monoclonal antibodies or monospecific polyclonal antibodies necessary to treat the affected individual will likely be greater than if the treatment regimen had begun early after the first signs of EHEC infection were detected. Treatment may also be warranted if a first individual who has shown no indications of EHEC infection is exposed to a second individual who has shown the clinical symptoms associated with an EHEC infection. This is especially true in cases where the individual is a child or an elderly person.

The therapeutic amount of antibody given to an individual suffering from HUS will be determined as that amount deemed effective in treating or ameliorating the disease. Normally, a monoclonal antibody, a cocktail of monoclonal antibodies or monospecific polyclonal antibodies will be administered in a pharmaceutically acceptable or compatible carrier. Therefore, the present invention also encompasses pharmaceutical compositions for the treatment of HUS, said compositions comprising a carrier and an effective amount of the monoclonal antibody, cocktail of monoclonal antibodies or monospecific polyclonal antibodies which specifically bind to either ST, SLT-I or SLT-II.

The pharmaceutical compositions are prepared by methods known to one of skill in the art. In general, a monoclonal antibody, a cocktail of monoclonal antibodies or monospecific polyclonal antibodies are admixed with a carrier and other diluents necessary to prepare the pharmaceutical composition, so that it is in a stable and administrable form. Administration of the pharmaceutical composition can be accomplished by several means. These means include, oral, intradermal, subcutaneous, intravenous or intramuscular.

The most efficient means of oral administration will require the pharmaceutical composition to take the form of a tablet or capsule. The tablet or capsule is designed such that dissolution and release of the monoclonal antibody, cocktail of monoclonal antibodies or monospecific polyclonal antibodies will not occur in the stomach. Instead, dissolution will be targeted to occur near to or directly at the site in the intestinal tract where EHEC has colonized. If the aforementioned tablet or capsule does not have these properties, they will need to be given with a solution capable of neutralizing stomach acid. One example of a solution capable of neutralizing stomach acid is sodium bicarbonate, though the present invention is not limited by disclosure of said solution. Application of a monoclonal antibody, a cocktail of monoclonal antibodies or monospecific polyclonal antibodies at the site of colonization will result in both neutralization of SLT at one of the primary sites of production and uptake of the antibodies into the blood stream leading to its dissemination to other sites in the body of the individual where SLT may be present.

If a capsule or tablet can not be created as a means for the oral ingestion of a monoclonal antibody, a cocktail of monoclonal antibodies or monospecific polyclonal antibodies, a second method of oral administration can be utilized. This method involves a less efficient means of oral administration wherein, a pharmaceutical composition is comprised of a monoclonal antibody, a cocktail of monoclonal antibodies or monospecific polyclonal antibodies admixed with an acid neutralizing solution prior to oral ingestion. The pharmaceutical composition is then orally ingested by the affected individual.

Other methods of administration require pharmaceutical compositions containing carriers that have been documented extensively in the prior art. These alternative methods of administration, intravenous, intramuscular, intradermal and subcutaneous administration can all be accomplished by admixing a monoclonal antibody, a cocktail of monoclonal antibodies or monospecific polyclonal antibodies with a balanced salt solution or its equivalents as the carrier. Selection of a particular balanced salt solution or its equivalents will be well known to one of skill in the art.

Purified SLT antigen is used to immunize animals for the production of monoclonal or polyclonal antibodies which bind specifically to either ST, SLT-I or SLT-II. Production of purified SLT antigen is described in detail in the following section. In general, the method takes advantage of the carbohydrate specificity of the toxin's binding domain. SLT binds specifically to the P₁-glycoprotein purified from hydatid cyst fluid. By coupling the P₁-glycoprotein to Sepharose 4B, a solid phase system for capturing toxin is generated. To purify SLT, a bacterial lysate containing either SLT-I or SLT-II is applied to a column containing the coupled matrix. Non-specifically and weakly binding material is washed off the column, followed by elution of the SLT with a buffer containing, for example, 4.5M MgCl₂. This method has resulted in yields of purified SLT that exceed 80% of the starting material applied to the column. In addition, the purified SLT material has been found to have very high specific activity (cytotoxin activity/mg protein). This scheme is improved over those disclosed in the prior art because it is capable of successfully purifying both SLT-I and SLT-II.

In one aspect of the present invention, human monoclonal and human monospecific polyclonal antibodies are produced by utilizing transgenic mice that are capable of expressing a diversity of human heavy and light chain immunoglobulins. These mice are described in more detail in the following section. The transgenic mice so used contain the heavy and light chain protein coding regions in an unrearranged configuration according to published procedures (Taylor et al., Nucl. Acid Res. 20:6287-6295 (1992)). To produce human monoclonal or human monospecific polyclonal antibodies with the appropriate specificity, transgenic mice are immunized repeatedly with either purified SLT-I or SLT-II. Following immunization of the transgenic mice, spleen cells are isolated and fused with myeloma cells, thus creating human monoclonal antibody cell lines. The specific methods used to produce hybridomas and monospecific polyclonal antibodies have been described in great detail in the prior art and would be known to one of skill in the art.

The most common method used to purify antigen specific polyclonal antibodies from immune serum is immunoaffinity purification on an antigen column. In this method pure antigen, in the present invention either SLT-I or SLT-II, is covalently coupled to a solid support. The immune polyclonal serum is passed through the column, and bound antibody eluted with either a high pH or low pH buffer as disclosed in Antibodies, A Laboratory Manual. Harlow and Lane, Cold Spring Harbor laboratory, 1988.

To determine the neutralizing activity of the ST, SLT-I and SLT-II human monoclonal or human monospecific polyclonal antibodies, tests can be carried out either in vitro in HeLa cells or in vivo in the piglet model (Tzipori et al., Infect. and Immun. 63:3621-3627, (1995)). Briefly, gnotobiotic piglets are challenged with E. Coli 0157:H7. At various intervals after inoculation, they receive the human monoclonal or human monospecific polyclonal antibodies at various concentrations to establish the optimal therapeutic dose required to protect them from developing severe neurological symptoms and death. After extensive quality, safety, reactogenicity, and efficacy studies in vitro and in various animal systems, the human monoclonal or human monospecific polyclonal antibodies are tested in human volunteers. Following this initial testing, the human monoclonal or human monospecific polyclonal antibodies are included in a pharmaceutical composition as described above to treat individuals suffering from HUS.

In addition, monoclonal antibodies which specifically bind ST, SLT-I or SLT-II can be produced by recombinant DNA methodology. Monoclonal antibody fragments (e.g. Fab fragments) can also be produced in this way. One means of doing this is through the production of a phage display library and the selection of clones with the appropriate specificity (Monoclonal Antibodies from Combinatorial Libraries, Cold Spring Harbor Course, (1993)). This method involves generation of heavy (V_{H}-C_{H1}) and light (V_{L}-C_{L}) chain genes in vitro by methods known to one of skill in the art. The library containing recombinantly produced monoclonal antibody (Fab) fragments is cloned into an M13 surface display vector or its equivalent and the resulting M13 phages or their equivalents, displaying anti-ST, anti-SLT-I or SLT-II antibody (Fab) fragments on their surface are screened and selected by bio-panning. The affinities of the monoclonal antibody (Fab) fragments selected by bio-panning can be further improved through DNA mutagenesis by conventional techniques. A large scale preparation is made from a purified single phage plaque, with said preparation used to either prepare phagemid DNA or purify the ST, SLT-I or SLT-II monoclonal antibody (Fab) fragments expressed on the surface of the M13 phage.

In a second aspect, the recombinant DNA methodology is used to produce chimeric monoclonal antibodies which specifically bind either ST, SLT-I or SLT-II. Chimeric monoclonal antibodies are created by excising the heavy (V_{H}) and the light (V_{L}) chain genes from the purified M13 phagemid DNA and cloning them into a human immunoglobulin expression vector. In this vector the human immunoglobulin constant regions are spliced to the 3′ end of the monoclonal antibody (Fab) fragment, generating a chimeric monoclonal antibody which in Example 3 of the following section yields a monkey-human chimeric or a mouse chimeric. The immunoglobulin expression vector containing the chimeric monoclonal antibody is transfected by electroporation into a cell line which is defective in Ig chain production.

Transformed cells containing the expression vector encoding the chimeric monoclonal antibody are isolated by conventional means. These cells are then grown in culture and their antibodies purified. Following testing by the methods described above for human monoclonal and monospecific polyclonal antibodies, the chimeric monoclonal antibodies can be used,for the therapeutic treatment of individuals suffering from HUS.

The present invention encompasses all monoclonal antibodies that can be generated which specifically bind either ST, SLT-I or SLT-II or their derivatives thereof. This includes those monoclonal antibodies generated with the appropriate specificity by techniques not specifically disclosed in the present Specification. In addition, the present invention encompasses monospecific polyclonal antibodies which specifically bind either ST, SLT-I or SLT-II or there derivatives thereof. Included are those monospecific polyclonal antibodies produced in mice capable of producing human antibody following immunization with either SLT-I or SLT-II.

### EXEMPLIFICATION

### EXAMPLE 1

Results

Oral inoculation of piglets with E. coli 0157:H7 strains

In the present study GB piglets were inoculated within 24 hours after birth with approximately 10¹⁰ viable EHEC 0157 organisms and were observed for symptoms over 5 days. Infected piglets normally develop symptoms of diarrhea within 2-3 days after challenge which continue for several days and results in wasting. Histologically, the mucosa of the terminal ileum and the large intestine are severely damaged due to bacterial A-E lesions mediated by the eaeA gene. Challenge of GB piglets with E. coli 0157:H7 strains 931, 3100-85, and 933, all SLT-I & II producers, normally lead to diarrhea and wasting, and some 25-30% of them go on to develop ED-like neurological symptoms (Table 1). In contrast, challenge with E. coli 0157:H7 strains 86-24 (Table 1) and RCH/86, both SLT-II producers, result in higher incidence of neurological symptoms and death, reaching 100% of animals. In particular, strain RCH/86 was isolated from a fatal case of bloody diarrhea, complicated with HUS and profound neurological symptoms. In piglets, diarrhea and neurological symptoms develop more rapidly with strain 86-24 than with 933.

TABLE 1
Summary of clinical and histological observations
in piglets inoculated with two EHEC and 2 control strains.

| Group | E. coli | Number of | Clinical outcome | | A-E |
|---|---|---|---|---|---|
| Number | Strain | animals | diarrhea | Neurol/coma/death# | lesions* |
| 1 | 86-24 (wild) | 8 | 8 | 8 | + |
| 5 | 933 (wild) | 16 | 16 | 5 | + |
| 6 | E. coli HS | 4 | 0 | 0 | − |
| 7 | K12 C600 | 2 | 0 | 0 | − |

#Piglets were autopsied within 40 to 72 hours after challenge, with onset of symptoms/death.
*Where (−) indicates no bacterial attaching-effacing (A-E) lesions were observed in any of the animals in this group; (+) indicates extensive lesions were observed in the large intestine and in the terminal ileum in all of them.

The intact gut epithelium forms a formidable barrier which keeps the bulk of SLT in the lumen where it is produced by bacteria in large quantities. Although small amount of SLT-II does get through as is demonstrated clinically in humans and experimentally in piglets, most of it remains in the lumen. A fraction however is taken up and remains bound within the gut mucosa as observed by immunohistochemistry (IHC) in frozen sections. In these sections the amount of mucosa-bound SLT-I is many fold higher than mucosa-bound SLT-II, indicating that SLT-I is “stickier” than SLT-II and could be the reason that it does not readily reach the circulation as does the less sticky SLT-II.

SLT-Specific Murine mAbs

Murine monoclonal antibodies (mAb) were raised against SLT-I and SLT-II. In a first attempt, characterization of 5 mAb cell lines produced against Shiga toxin using immunoprecipitation yielded 3 with the appropriate specificity. One example is 4D3, an IgG mAb specific for the B subunit, which neutralized SLT-I very effectively when preincubated with toxin before addition to the HeLa cells. If toxin was prebound to cells first, the antibody had no significant protective effect. In contrast, the other two mAbs which recognized an epitope on the A subunit, showed less dramatic neutralization when preincubated with toxin before addition to HeLa cells. However, these two mAbs were highly protective when added to cells that were prebound with toxin. All 3 mAbs were IgG1. In a second attempt, mAbs were generated against SLT-II. In this study eight hybridomas were isolated and 4 were characterized. The two B subunit specific mAbs strongly neutralized SLT-II cytotoxicity to HeLa cells. One of these mAbs also cross-reacted with the SLT-I B subunit and was able to neutralize SLT-I cytotoxicity. The two A subunit mAbs had no neutralizing activity and failed to react against the toxin in solution, but reacted with coated toxin on ELISA plates. The two A subunit specific mAbs were IgM and the two B subunit specific mAbs were IgG, one being IgG1 and the other IgG2b.

Challenge and Protection of GB Piglets

Subsequent to challenge with SLT-II producing E. coli 0157:H7, GB piglets were treated with specific antibodies. Table 2 summarizes the outcome of the challenge-protection experiment, in which 7 of the 8 control animals developed neurological symptoms and died within 72 hours. The animal which did survive, suffered episodes of seizure that lasted several seconds. Of the animals given SLT-II pig immune serum, a total of 3 developed neurological symptoms (1 of 6 from the 12 hour group and 2 of 6 from the 24 hour group). Characteristic discreet hemorrhages in the cerebellum associated with the disease were observed only in the 6 euthanized control piglets. It is not clear why the 3 piglets which developed neurological symptoms despite being given only the immune serum had no such cerebellar lesions. All animals that were challenged had A-E lesions in the colon.

TABLE 2
Survival of GB piglets infected with 10¹⁰
organisms of E. coil 0157:H7 strain 86-24, 24 hours after
birth. At 6, 12 or 24 after challenge, piglets were
injected intraperitoneally (IP) with either 4 ml/kg of SLT-II
pig immune serum, or with control pig serum. They were
monitored for survival over 72 hours after challenge.

| | | Number of | | |
|---|---|---|---|---|
| | | animals | | |
| Serum given* | Number of | Survived | Hemorrhages | A-E |
| after challenge | Animals | 72 hours | in cerebellum | Lesions |
| No serum given | 2 | 0 | 2 | 2 |
| 12 hr. (control serum) | 6 | 1# | 6 | 6 |
| 6 hr. (SLT serum) | 2 | 2 | 0 | 2 |
| 12 hr. (SLT serum) | 6 | 5 | 0 | 6 |
| 24 hr. (SLT serum) | 6 | 4 | 0 | 6 |

*SLT-II immune serum was produced in a two-months old pig given 4 consecutive intramuscular injections with affinity-purified SLT-II. The control serum was from unimmunized animal.
#One surviving piglet displayed occasional fits but survived.

This experiment shows that piglets can be protected from the systemic effect of SLT and death with specific antitoxin neutralizing antibodies, even when given well after the bacterial challenge. In this animal model, the piglets present clinical symptoms approximately 48 hours after challenge, which is a shorter time period than humans. The results that have been presented are significant and suggest that children could likewise be protected against development of renal failure and other systemic complications, if treated early with neutralizing SLT-specific antibodies. This is likely to be at the onset of bloody diarrhea or with confirmed infections with SLT-producing bacteria. The benefit of antibody administration earlier to sibling of affected individuals, or in an outbreak in a day-care setting, will be much greater. Systemic administration of SLT antibody however did not protect piglets from developing mucosal lesions of A-E and diarrhea. This experiment confirms our hypothesis that treatment with highly specific neutralizing antibodies, even when given after exposure, is very likely to be beneficial. Since the half-life of exogenous Ig in humans is reported to range between 6 and 14 days, probably a single effective dose might be sufficient. Using human mabs however, multiple injections, if need be, should be reasonably safe. This might occur if plasma exchange is applied.

Materials and Methods

Toxin purification and toxoid production

Hydatid cysts isolated from sheep infected Echinococcus granulosus contain material, identified as a glycoprotein, which has P₁ blood group reactivity. The P₁ glycoprotein's antigenic determinant was subsequently shown to consist of a trisaccharide, Gala-4GalB1-4GlcNAc, identical to the non-reducing end of the P₁ glycolipid on human erythrocytes. Shiga toxin, SLT-I and -II bind to terminal Galα1-4Gal disaccharide of glycolipids and hence, the P1-glycolipid is a receptor for these toxins. The P₁ glycoprotein in hydatid cyst fluid interacts directly with Shiga toxin and inhibits Shiga toxin binding and cytotoxicity to tissue culture cells. By covalently coupling the hydatid cyst glycoprotein to Sepharose 4B a solid phase system for capturing toxin is generated. To purify SLT-I, C600(933J) is grown in low syncase medium in the presence of 200 ng/ml of mitomycin C. Mitomycin C induces the 933J bacteriophage carrying the genes for SLT-I. For the purification of SLT-II strain C600(933W) is grown in LB broth in the presence of 200 ng/ml mitomycin C. The toxin from both strains is found predominately in the culture supernatant and the approximate yields are 5 mg/liter for SLT-I and 10 mg/liter for SLT-II. A 70% ammonium sulfate precipitation of the culture supernatant is made and the precipitate dissolved in 10 mM Tris (pH 7.4) and dialyzed against the same buffer. To further purify SLT-I and -II, bacterial lysate is applied to a column containing the coupled matrix. To remove non-specifically or weakly attached proteins, the column is washed with buffer containing 1 M NaCl and finally toxin is eluted with buffer containing 4.5 M MgCl₂. For long term storage the eluted protein is dialyzed extensively against 20 mM ammonium bicarbonate, lyophilized and stored at −70 C. This method results in an increase in specific activity (cytotoxin activity/mg protein) of more than 1000 fold, with yields of toxin greater than 80%. in addition to the purification of SLT-I and -II, both the SLT-IIe, the toxin involved in edema disease in pigs and a SLT-II variant from a human isolate have been purified. To immunize either GB piglets or the human monoclonal antibody (HuMAb) mice, toxin will be inactivated by treatment with 4% paraformalaldehyde at 37° C. for two days after which the fixative will be removed by overnight dialysis with PBS. The degree of inactivation will be comparing HeLa cell cytotoxicity of the toxoid to the untreated toxin.

Piglet EHEC Challenge and Protection Model

Twenty-two GB piglets were challenged with a high dose of 10¹⁰ EHEC 0157 to ensure that 100% of animals develop fatal neurological symptoms within 40-72 hours. They were then divided into 5 uneven groups as shown in Table 2. One control group remained untreated, while the second was given 12 hours after bacterial challenge 4 ml/kg IP of serum from normal unimmunized pig. Groups 3-5 were similarly given 4 ml/kg IP of SLT-II specific pig immune serum 6, 12, or 24 hours after challenge, respectively. The SLT-II immune pig serum was collected from a weaned pig which was given 4 consecutive intramuscular injections of affinity-purified SLT-II, and stored in aliquots at −70° C.

Assay of SLT II GB Piglet Immune Sera

Toxin (b 100 pg/ml) was reincubated for 1 h at room temperature with dilutions of either the pig immune serum or dilutions of mouse ascites fluid containing 4D1 mAb. The pretreated toxin was then added to 96 well tissue culture plates containing HeLa cell monolayers. Each mixture of toxin/antibody concentration was added in triplicate. Following overnight incubation at 37° C. the wells were washed and the remaining cells stained by crystal violet, washed and absorbance read at 595 nm. The medium control is used as the 100% survival level.

### EXAMPLE 2

Results

Human Monoclonal Antibody Production

The human monoclonal antibody (HuMAb) transgenic mouse strain used in the present invention contains the 80-kilobase (kb) heavy chain construct, pHC2, which encodes 4 variable (Vh), 15 diversity (Dh) and 6 joining (Jh) segments along with the μ and γ1 C exons together with their switch regions, the Jh intronic enhancer and the rat 3′ heavy chain enhancer. The light chain transgene, pKCo4, is derived from the co-integration of two DNA fragments, one fragment comprising 4 Vk segments and the other fragment the 3′ Vk segment of the first fragment along with the 5 Jk segments, the Ck exon, the intronic enhancer and the downstream enhancer. A new transgenic mouse strain was generated that contained additional Vk segments. This was accomplished by the co-injection of the pKCo4 mini-locus with a yeast artificial chromosome clone that includes the distal half of the human Vk gene segments. The disruptions of the endogenous murine heavy and κ light chain immunoglobulin loci were accomplished by replacing segments of those loci with the neomycin resistance gene through homologous recombination. The Jh segments were replaced in the heavy chain mutant, and the Jk segment and Ck exon were targeted in the κ light chain mutant, both of which prevent VDJ (or VJ) rearrangement and subsequent expression of murine immunoglobulin.

Both of the transgenes were microinjected into mouse embryo pronuclei and transgene expressing founder animals were obtained. The targeted deletions of the murine heavy chain and kappa light chain loci were attained with appropriate vectors in mouse embryonic stem (ES) cells. ES clones carrying these disruptions were injected into blastocysts to produce chimeras which were then bred to ultimately generate mice homozygous for either murine immunoglobulin chain deletion. The double transgenic, double deletion HuMAb mice were obtained by breeding of the transgene-positive animals with the mice carrying the targeted deletions. These double transgenic, double deletion mice have B cells which can express a human Ig receptor, develop in the bone marrow and populate peripheral lymphoid organs.

Antibodies generated in mice containing rearranged transgenes use essentially all of the V and J segments present in the transgene. Moreover, the HuMAb mice undergo class switching as evidenced by the initial IgM response followed by a human IgG response to immunogen. This has been confirmed to be authentic class switching by genomic recombination between the transgene μ and γ1 switch regions in mice which carried only the Jh deletion and a human heavy chain transgene with fewer Vh and Dh segments. Consequent with the class switch is extensive somatic mutation of the human heavy chain V regions.

Even with their limited heavy and light chain transgene repertoire, the human monoclonal antibody transgenic mice have responded by producing human IgM and IgG to all haptens and antigens that have been tested to date. These antigens and haptens include human CD4, human IgE, human TNF, human lymphocytes, human RBC, human carcinoma cells, CEA, KLH, and DNP. Human IgM and IgG antigen-specific mAb have been produced from both the original strain and the subsequent strain containing additional Vk gene segments of these immunized mice following standard hybridoma production procedures. Because the HuMAb transgenic mice have 10-50% of the normal level of B cells, isolating human mAb-producing hybridomas does require slightly more effort than when producing murine mAbs. Multiple mice need to be immunized and their sera screened. Generally, 20-80% of the mice respond to a given antigen with sufficiently high titers to be candidates for fusion. To date, approximately half of the fusions performed have led to the isolation of stable human IgM-and IgG-secreting hybridomas. On average, between 1 and 12 hybridomas are obtained per fusion.

Materials and Methods

Immunization of Human Monoclonal Antibody Mice

Cumulative experience with a wide variety of antigens has shown that the transgenic mice used in the present invention respond best when immunized IP on day 1 with 20 to 100 μg of antigen in complete Freund's adjuvant followed by weekly IP immunizations (up to a total of 10, with a 2 to 5 week rest after weeks 5) with 5 to 20 μg of antigen in incomplete Freund's adjuvant. Mice were immunized with either SLT-I or SLT-II toxoid.

The immune response was monitored over the course of the immunization protocol (including pre-immunization), with serum samples being obtained by retro-orbital bleeds. The sera were serially diluted starting from 1:10 and screened in an ELISA using human γ-, μ-and Kκ-specific secondary antibodies for their reactivity with the SLT antigens.

This method did not exclude the use of other adjuvants such as TiterMax, other routes of immunization such as subcutaneous, or other transgenic strains of HuMAb mice. Transgenic mice breed very well and it was no problem to produce the numbers of mice needed for this project. Those mice with the highest titers of human immunoglobulin directed against the SLT antigens received 5 to 20 mg of antigen in saline IV 3 days before sacrifice and removal of the spleen.

Hybridoma Generation and Screening

The mouse splenocytes were isolated and fused with PEG to a mouse myeloma cell line based upon standard protocols known to one of skill in the art. The resulting mouse hybridomas producing human monoclonal antibodies were then screened for the production of antigen-specific antibodies. Specifically, single cell suspensions of splenic lymphocytes from immunized mice were fused to one-sixth the number of P3X63-Ag8.653 nonsecreting mouse myeloma cells (ATCC CRL 1580) with 50% PEG (Sigma). Cells were plated at approximately 2×10⁵ in flat bottom microtiter plates, followed by a two week incubation in selective medium containing 20% Fetal Clone Serum (HyClone), 18% “653” conditioned medium, 5% Origen (IGEN), 4 mM L-glutamine, 1 mM sodium pyruvate, 5mM HEPES, 0.055 mM 2-mercaptoethanol, 50 units/ml penicillin, 50 mg/ml streptomycin, 50 mg/ml gentamycin and 1×HAT (Sigma; the HAT was added 24 hr. after the fusion). After two weeks, cells were cultured in medium in which the HAT is replaced with HT. Individual wells were then screened by ELISA for human IgM and IgG anti-SLT mAbs once extensive hybridoma growth or spent medium is observed, usually after 10-14 days. The hybridomas secreting such antibodies were replated, screened again, and, if still positive for human IgM and IgG anti-SLT monoclonal antibodies, subcloned at least twice by limiting dilution. The stable subclones were then cultured in vitro to generate small amounts of antibody in tissue culture medium for the initial characterizations. The most interesting monoclonal antibodies were produced in large quantities by growth of the hybridomas as ascites cells in nude mice. Should the occasional hybridoma fail to generate ascites fluid, it was expanded in vitro instead in cultures up to 1 liter and mAb purified over a Protein A column from the spent cell culture supernatant. A typical hybridoma will produce up to 10 mg of mAb from tissue culture or ascites with >60% purity.

Screening for SLT-Positive Hybridomas

Two methods were used to screen the hybridoma supernatants. Supernatants were screened by both ELISA, using plates coated with purified SLT II and by immunoprecipitation, using radiolabeled SLT II. The reason for this two-fold screening procedure was to maximize the chance of finding mAbs which interact with and neutralize toxin, since not all monoclonal antibodies which interact with toxin coated on an ELISA plate were able to interact with toxin in solution and immunoprecipitate ¹²⁵I-labeled toxin.

ELISA of SLT-Specific Hybridoma Serum

Nunc-Immuno Plates Maxi-Sorp were coated overnight at 4° C. with either 100 μl of SLT-I or 100 μl SLT-II (1 μg/μl for each toxin) in PBS and then blocked at room temperature for 1 hour with 1% BSA in PBS to saturate nonspecific protein binding sites. Plates were washed extensively with PBS-Tween 20 (0.05%) prior to the addition of hybridoma supernatants (40 82 l/well). The supernatants were incubated for 2 h at room temperature. After washing again with PBS-Tween, the plates were developed with a goat anti-human Ig polyvalent antibody conjugated with alkaline phosphatase and phosphatase substrate. The A₄₀₅ was measured using an automated ELISA reader.

Immunoprecipitation Studies

In screening the hybridoma supernatants, radio-labeled antigen was immunoprecipitated. Immunoprecipitation studies were done to look at both the ability to precipitate the specific immunogenic toxoid molecule and the ability to cross-react with the two toxoid molecules. Using purified antibody, an immunoprecipitation titer of the hybridoma supernatant was generated by mixing a fixed quantity of antigen with varying amounts of the mAb. When a monoclonal antibody cross-reacts against both SLT-II and -I, the curves of cpm immunoprecipitated versus antibody concentration was given as an estimate to the degree of cross-reaction. Toxin was labeled by Chloramine T iodination. To assay culture supernatants, 20 microliters of supernatant was mixed with 50,000 cpms of ¹²⁵I-labeled toxin (˜1 ng toxin) to a volume of 100 microliters. After 1 h at room temperature, a rabbit anti-human IgG1 was added to ensure efficient immunoprecipitation. Antibody-toxin complexes were immunoprecipitated using fixed protein A-positive Staphylococcus aureus by the procedures known to one of skill in the art. An irrelevant Hu/mAb was used as the negative control for all the laboratory assays.

### EXAMPLE 3

Construction of Monoclonal Antibodies by Creation of a Phage

Display Library

The anti SLT-I and SLT-II antibodies are generated by phage surface display technology as follows: In this approach, a library of Heavy (V_{H}-C_{H1}) and Light (V_{L}-C_{L}) chain genes are generated in vitro. This library is cloned into an M13 surface display vector (pComb3 or its equivalent) and the resulting M13 phages, displaying anti SLT I and SLT II antibodies on their surface, are screened and selected by bio-panning. ps Materials and Methods

Enrichment of lymphocytes secreting anti SLT I and anti SLT II antibodies

Lymphocytes secreting anti SLT-I and anti SLT-II antibodies are enriched according to Linton et al. (Linton et al., Cell 59:1049-1059 (1989)). Purified lymphocytes are incubated for 45 minutes with 60 nM biotin-SLT-I or biotin-SLT-II toxin, washed twice, and then poured onto petridishes coated with streptavidin and blocked with bovine serum albumin, incubated for another 60 minutes at 4° C., and then washed extensively. After the last wash, the petridishes are shaken dry and the bound cells are used for the isolation of total RNA.

Preparation of total RNA

Total RNA is prepared either from purified lymphocytes or from purified and enriched lymphocytes by the modified Chomczynski and Sacchi method (Chomczynski and Sacchi, Anal. Biochem. 162:156-159 (1987)). 2 mls RNAzol (Biotecx) per 10-100 mgs of cells is added and the total RNA is isolated according to the manufacturers' recommendation. The total RNA is precipitated with isopropanol and washed with 70% ethanol and resuspended in TE buffer made with DEPC treated water.

Synthesis of cDNA and PCR amplification of Heavy (V_{H}-CH_{H1}) and Light (V_{L}-C_{L}) chains

Monkey heavy and light chain cDNAs are synthesized according to Barbas and Burton (Barbas and Burton, Monoclonal Antibodies from Combinatorial Libraries: Cold Spring Harbor Laboratory Course (1993)). 1 μl (10-30 gg) of total RNA is mixed with 1 μl (60 pmoles) of heavy or light chain 3′ primer or oligo dT and 5 μl of DEPC treated water. The mixture is heated to 70° C. and cooled slowly. 5 μl of 5×RT buffer, 2 μl of 10 mM dNTP mixture, 0.5 μl of RNasin, 0.5 μl (200 units) of MMLV Reverse Transcriptase and 5 μl of DEPC treated water are added to the sample and incubated at 37° C. for 45 minutes. The resulting cDNA is used in further DNA amplifications using 5′ and 3′ heavy and light chain amplifiers in the standard PCR protocols. The PCR primers used in the amplification of heavy and light chains have the following restriction sites that allow the double stranded PCR product to be cloned into the pComb3 vector.

| | |
|---|---|
| 5′ Heavy chain primer | CTCGAG XhoI |
| 3′ Heavy chain primer | ACTAGT SpeI |
| 5′ Light chain primer | GAGCTC SacI |
| 3′ Light chain primer | TCTAGA XbaI |

Cloning and expression of the synthetic antibodies (Fab), on the surface of M 13 bacteriophage

Heavy (V_{H}-C_{H1}) and light (V_{L}-C_{L}) chain DNAs are amplified using appropriate PCR primers and the cDNA made from the lymphocytes. The amplified double stranded DNA is electrophoretically purified on agarose gels. The purified DNA band (2-5 Ag) is cut with suitable restriction enzymes and ligated in pComb3 vector. The ligation mixture is ethanol precipitated and washed with 70% ethanol and air dried. The pellet is dissolved in 10 μl TE. 1-2 μL is used to electroporate XL-1 Blue cells. Transformants are grown at 37° C., in LB amp. After one hour of growth, helper phage VCSM13 is added (10¹² pfu) and grown for an additional 2 hours. 50 μg/μl of kanamycin is added and the culture is grown o/n at 37° C. M13 phage is prepared from the culture supernatant by standard procedures and is used in bio-panning.

Bio-panning

96 well ELISA plates are coated with 25 μL of either SLT I or SLT II (0.5-0.1 μg/well) in PBS. The plates are incubated at 4° C. for 12 hours. The coating solution is removed and the plates are washed twice with deionized water. After removing the residual water, the plates are blocked with 3% BSA in PBS for 1 hour at 37° C. After removing the 3% BSA solution, 50 μL of phage suspension (approximately (10¹² pfu) is added to each well and the plates are incubated at 37° C. for 2 hours. At the end, the phage is removed and plates are washed vigorously with TBS/0.5% (TBST). The bound phages are eluted with elution buffer (0.1 M HC1, pH 2.2, adjusted with glycine). This bio-panning is repeated at least three times, with increasing stringency at the wash step and the bound phages are eluted with elution buffer. A large scale phage preparation is made from a purified single phage plaque and the phagemid DNA is prepared. Heavy (V_{H}-C_{H1}) and light (V_{L}-C_{L}) chain gene sequences from this plasmid are analyzed. Subsequently, only the variable regions of the heavy (V_{H}) and the light (V_{L}) chain genes are cloned in a human immunoglobulin expression vector. In this vector, the human immunoglobulin constant regions are spliced at the 3′ end of the synthetic monkey variable region, generating a synthetic, monkey-human chimeric antibody gene.

Expression and purification of recombinant, monkey-human hybrid anti SLT I and anti SLT II antibodies

The immunoglobulin expression vector containing the chimeric antibody gene is transfected into mouse myeloma cell line (ATCC CRL 1580), which is defective in IgG chain, by electroporation. After incubation on ice for 10 minutes, the cells are transferred to 20 mls of culture medium and incubated at 37° C. for 48 hours in a CO₂ incubator. Cells are plated in a 96 well microtiter plates at density of 2×10⁴. Cells from the master wells secreting the most antibody are subjected to limiting dilution and are plated. Antibodies from the culture supernatant are purified and used in animal studies.

1. A therapeutic method to treat hemolytic uremic syndrome in an individual, said method comprising:
a) providing a monoclonal antibody which binds specifically to Shiga like toxin; and
b) administering the monoclonal antibody to a mammal in a therapeutically effective amount.

2. The therapeutic method of claim 1, wherein the monoclonal antibody binds specifically to Shiga like toxin I.

3. The therapeutic method of claim 1, wherein the monoclonal antibody binds specifically to Shiga like toxin II.

4. The therapeutic method of claim 1, wherein the monoclonal antibody is a human monoclonal antibody.

5. The therapeutic method of claim 1, wherein the monoclonal antibody is produced by recombinant DNA methodology.

6. The therapeutic method of claim 1, wherein the monoclonal antibody is a chimeric monoclonal antibody.

7. The therapeutic method of claim 1, wherein the hemolytic uremic syndrome is caused by a Shiga-like toxin producing bacteria.

8. The therapeutic method of claim 7, wherein the Shiga-like toxin producing bacteria is Enterohemorrhagic Escherichia coli.

9. A monoclonal antibody which binds specifically to Shiga like toxin I.

10. The monoclonal antibody of claim 9, which is a human monoclonal antibody.

11. The monoclonal antibody of claim 9, which is produced by recombinant DNA methodology.

12. The monoclonal antibody of claim 9, which is a chimeric monoclonal antibody.

13. A monoclonal antibody which binds specifically to Shiga like toxin II.

14. The monoclonal antibody of claim 13, which is a human monoclonal antibody.

15. The monoclonal antibody of claim 13, which is produced by recombinant DNA methodology.

16. The monoclonal antibody of claim 13, which is a chimeric monoclonal antibody.

17. A therapeutic method to treat hemolytic uremic syndrome in an individual, said method comprising:
a) providing monospecific polyclonal antibodies which bind specifically to Shiga like toxin; and
b) administering the monospecific polyclonal antibodies to a mammal in a therapeutically effective amount.

18. The therapeutic method of claim 17, wherein the monospecific polyclonal antibodies bind specifically to Shiga like toxin I.

19. The therapeutic method of claim 17, wherein the monospecific polyclonal antibodies bind specifically to Shiga like toxin II.

20. The therapeutic method of claim 17 wherein the monospecific polyclonal antibodies are human monospecific polyclonal antibodies.

21. The therapeutic method of claim 17, wherein the hemolytic uremic syndrome is caused by a Shiga-like toxin producing bacteria.

22. The therapeutic method of claim 21, wherein the Shiga-like toxin producing bacteria is Enterohemorrhagic Escherichia coli.

23. Monospecific polyclonal antibodies which bind specifically to Shiga like toxin I.

24. The monospecific polyclonal antibodies of claim 23, which are human monospecific polyclonal antibodies.

25. Monospecific polyclonal antibodies which bind specifically to Shiga like toxin II.

26. The monospecific polyclonal antibodies of claim 25, which are human monospecific polyclonal antibodies.
